(19) United States
(12) Patent Application Publication
(10) Pub. No.: US 2014/0343063 A1
(43) Pub. Date: Nov. 20, 2014

(54) METHOD OF TREATING A VIRAL INFECTION USING ELVITEGRAVIR COMBINATIONS

(71) Applicant: Gilead Sciences, Inc., Foster City, CA (US)

(72) Inventors: Brian P. KEARNEY, San Francisco, CA (US); Anita A. MATHIAS, Sunnyvale, CA (US); Srinivasan RAMANATHAN, San Carlos, CA (US)

(73) Assignee: GILEAD SCIENCES, INC., Foster City, CA (US)

(21) Appl. No.: 14/281,798

(22) Filed: May 19, 2014

Related U.S. Application Data
(63) Continuation of application No. 12/147,041, filed on Jun. 26, 2008, now abandoned.
(60) Provisional application No. 60/947,306, filed on Jun. 29, 2007.
(60) Provisional application No. 61/040,920, filed on Mar. 31, 2008.

(51) Int. Cl. A61K 31/5377; A61K 31/4402; A61K 31/427; A61K 31/47

(52) U.S. Cl. CPC A61K 31/5377; A61K 31/47; A61K 31/4402; A61K 31/427
USPC 514/236.8; 514/312

(57) ABSTRACT

The invention includes methods, compositions, and kits useful for treating a viral infection by administering 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, with atazanavir or a pharmaceutically acceptable salt thereof, and optionally with a compound that inhibits cytochrome P-450, or a pharmaceutically acceptable salt thereof.

### PRIORITY OF INVENTION

This application is a continuation application of U.S. patent application Ser. No. 12/147,041, filed Jun. 26, 2008, which claims priority from U.S. Provisional Application No. 60/947,306, filed Jun. 29, 2007 and from U.S. Provisional Application No. 61/040,920, filed Mar.31, 2008. The entire content of each of which is hereby incorporated herein by reference in its entirety.

### BACKGROUND OF THE INVENTION

A series of 4-oxoquinolines including the compound 6-(3-chloro-2-fluorobenzyl)-1-[(2S)-1-hydroxy-3-methylbutan-2-yl]-7-methoxy-4-oxo-1,4-dihydroquinoline-3-carboxylic acid (the Compound) have been identified as anti-human immunodeficiency virus (HIV) agents. See U.S. patent application Ser. No. 10/492,833, filed Nov. 20, 2003, which was published as U.S. Patent Application Publication Number 20050239819. Specifically, the Compound has been described as having inhibitory activity against the integrase protein of HIV. Id. HIV belongs to the retrovirus family and is a causative agent of the acquired immunodeficiency syndrome (AIDS). Accordingly, a pharmaceutical agent that reduces the virus load, viral genome, or replication of HIV in the body, may be effective for the treatment or prophylaxis of AIDS.

The treatment cost and the potential for unwanted side-effects can both increase as the required dose of a drug increases. Therefore, there is a need for methods and compositions that are useful for achieving an acceptable anti-viral effect using a reduced dose of the Compound.

### SUMMARY OF THE INVENTION

It has been determined that the systemic exposure to the Compound in humans improves when the Compound is administered with atazanavir (ATV) either with or without the co-administration of ritonavir. A dose of 300 mg of the Compound administered with atazanavir was found to have a systemic exposure equivalent to the 300 mg dose of the Compound upon co-administration with ritonavir. Additionally, a dose of 85 mg of the Compound administered with ritonavir-boosted atazanavir was found to have a systemic exposure equivalent to the 150 mg dose of the Compound alone.

Accordingly, in one embodiment the invention provides a method of treating a viral infection in a human comprising administering 1) 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; and 2) a compound that inhibits a UGT pathway or UGT metabolism, or a pharmaceutically acceptable salt thereof to the human. In one embodiment of the invention, this method further comprises administering a compound that inhibits cytochrome P-450, or a pharmaceutically acceptable salt thereof to the human.

The invention also provides a pharmaceutical composition comprising 1) 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; 2) a compound that inhibits a LICIT pathway or UGT metabolism, or a pharmaceutically acceptable salt thereof; and 3) a pharmaceutically acceptable carrier or diluent.

In one embodiment, the invention provides a kit comprising: (1) 6-(3-chloro-2-fluorobenzyl)-1-1-[(2S)-1-hydroxy-3-methylbutan-2-yl]-7-methoxy-4-oxo-1,4-dihydroquinoline-3-carboxylic acid, or a pharmaceutically acceptable salt thereof; (2) a compound that inhibits a UGT pathway or UGT metabolism, or a pharmaceutically acceptable salt thereof; (3) one or more containers; and (4) prescribing information regarding administering the 6-(3-chloro-2-fluorobenzyl)-1-[(2S)-1-hydroxy-3-methylbutan-2-yl]-7-methoxy-4-oxo-1,4-dihydroquinole-3′ carboxylic acid or a pharmaceutically acceptable salt thereof with the compound that inhibits a UGT pathway or UGT metabolism, or the pharmaceutically acceptable salt thereof.

In one embodiment, the invention provides a kit comprising: (1) a unit dosage form comprising 6-(1-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof; (2) a compound that inhibits a UGT pathway or UGT metabolism, or a pharmaceutically acceptable salt thereof; (3) one or more containers; and (4) prescribing information regarding administering the 6-(3-chloro-2-fluorobenzyl)-1-[(2S)1-hydroxy-3-methylbutan-2-yl]-7-methoxy-4-oxo-1,4-dihydroquinoline-3-carboxylic acid or a pharmaceutically acceptable salt thereof with the compound that inhibits a UGT pathway or UGT metabolism, or the pharmaceutically acceptable salt thereof.

### DETAILED DESCRIPTION OF THE INVENTION

As used herein, the term “coadminister” refers to administration of two or more agents within a 24 hour period of each other, for example, as part of a clinical treatment regimen. In other embodiments, “coadminister” refers to administration within 2 hours of each other, in other embodiments, “coadminister” refers to administration within 30 minutes of each other. In other embodiments, “coadminister” refers to administration within 15 minutes of each other, In other embodiments, “coadminister” refers to administration at the same time, either as part of a single formulation or as multiple formulations that are administered by the same or different routes.

The term “unit dosage form” refers to a physically discrete unit, such as a capsule, tablet, or solution that is suitable as a unitary dosage for a human patient., each unit containing a predetermined quantity of one or more active ingredient(s) calculated to produce a therapeutic effect, in association with at least one pharmaceutically acceptable diluent or carrier, or combination thereof.

If desired, the effective daily dose of the Compound may be administered as two three, four, five, six, or more sub-doses administered separately at appropriate intervals throughout the day, optionally, in unit dosage forms.

The concentration of the Compound in the bloodstream may be measured as the plasma concentration (e.g., ng/mL). Pharmacokinetic parameters for determining the plasma concentration include, but are not limited to, the maximum observed plasma concentration (Cₘₐₓ), observed plasma concentration at the end of the dosing interval or “trough” concentration (Cₜₐᵤ or Cₘᵢₙ), area wider the plasma concentration time curve (AUC) from time zero up to the last quantifiable time point (AUC₀₋ₗₐₛₜ), AUC from time zero to infinity (AUC_{0-inf}), AUC over the dosing interval (AUCtauₜₐᵤ), time of maximum observed plasma concentration after administration (tₘₐₓ), and half-life of the Compound in plasma (t_{1/2}).

Administration of the Compound with food according to the methods of the invention may also increase absorption of the Compound. Absorption of the Compound may be measured by the concentration attained in the bloodstream over time after administration of the Compound. An increase in absorption by administration of the Compound with food may also be evidenced by an increase in Cₘₐₓ and/or AUC of the Compound as compared to the values if the Compound was administered without food. Typically protease inhibitors are administered with food.

The present invention also provides a method for the treatment or prophylaxis of diseases, disorders, and conditions. An example of a disease, disorder, or condition includes, but is not limited to, a retrovirus infection, or a disease, disorder, or condition associated with a retrovirus infection. Retroviruses are RNA viruses and are generally classified into the alpharetrovirus, betaretrovirus, deltaretrovirus, epsilonretrovirus, gammaretrovirus, lentivirus, and spumavirus families. Examples of retroviruses include, but are not limited to, human immunodeficiency virus (HIV), human T-lymphotropic virus (HTLV), rous sarcoma virus (RSV), and the avian leukosis virus. In general, three genes of the retrovirus genome code for the proteins of the mature virus: gag (group-specific antigen) gene, which codes for the core and structural proteins of the virus; pol (polymerase) gene, which codes for the enzymes of the virus, including reverse transcriptase, protease, and integrase; and env (envelope) gene, which codes for the retrovirus surface proteins.

Retroviruses attach to and invade a host cell by releasing a complex of RNA and the poi products, among other things, into the host cell. The reverse transcriptase then produces double stranded DNA from the viral RNA. The double stranded DNA is imported into the nucleus of the host cell and integrated into the host cell genome by the viral integrase. A nascent virus from the integrated DNA is formed when the integrated viral DNA is converted into mRNA by the host cell polymerase and the proteins necessary for virus formation are produced by the action of the virus protease. The virus particle undergoes budding and is released from the hest cell to form a mature virus.

The active agents may be administered to a human in any conventional, manner. While it is possible for the active agents to be administered as raw compounds, they are preferably administered as a pharmaceutical composition. A “pharmaceutical composition comprising the Compound” refers to a pharmaceutical composition comprising the Compound, or a pharmaceutically acceptable salt thereof, with one or more pharmaceutically acceptable carriers or diluents and optionally other therapeutic agents and/or components. The salt, carrier, or diluent should be acceptable in the sense of being compatible with the other ingredients and not deleterious to the recipient thereof. Examples of carriers or diluents for oral administration include cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), and sodium lauryl sulfate.

The pharmaceutical compositions may be prepared by any suitable method, such as those methods well known in the art of pharmacy, for example, methods such as those described in Gennaro et al., Remington's Pharmaceutical Sciences (18th ed., Mack Publishing Co., 1990), especially Part 8: Pharmaceutical Preparations and their Manufacture. Such methods include the step of bringing into association the Compound with the carrier or diluent and optionally one or more accessory ingredients. Such accessory ingredients include those conventional in the art, such as, fillers, binders, excipients disintegrants, lubricants, colorants, flavoring agents, sweeteners, preservatives (e.g., antimicrobial preservatives), suspending agents, thickening agents, emulsifying, agents, and/or wetting agents.

The pharmaceutical compositions may provide controlled, slow release, or sustained release of the agents the Compound) over a period of time. The controlled, slow release, or sustained release of the agents (e.g. the Compound) may maintain the agents in the bloodstream of the human for a longer period of time than with conventional formulations. Pharmaceutical compositions include, but are not limited to, coated tablets, pellets, solutions, powders, and capsules, and dispersions of the Compound in a medium that is insoluble in physiologic fluids or where the release of the therapeutic compound follows degradation of the pharmaceutical composition due to mechanical, chemical, or enzymatic activity.

The pharmaceutical composition of the invention may be, for example, in the form of a pill, capsule, solution, powder, or tablet, each containing a predetermined amount of the Compound. In an embodiment of the invention, the pharmaceutical composition is in the form of a tablet comprising the Compound and the components of the tablet utilized and described in the Examples herein.

For oral administration, fine powders or granules may contain diluting, dispersing, and or surface active agents and may be present, for example, in water or in a syrup, in capsules or sachets in the dry state, or in a nonaqueous solution or suspension Wherein suspending agents may be included, or in tablets wherein binders and lubricants may be included.

When administered in the form of a liquid solution or suspension, the formulation may contain the Compound and purified water. Optional components in the liquid solution or suspension include suitable sweeteners, flavoring agents, preservatives (e.g., antimicrobial preservatives), buffering agents, solvents, and mixtures thereof. A component of the formulation may serve more than one function. For example, a suitable buffering agent also may act as a flavoring agent as well as a sweetener.

Suitable sweeteners include, for example, saccharin sodium, sucrose, and mannitol. A mixture of two or more sweeteners may be used. The sweetener or mixtures thereof are typically present in an amount of from about 0.001% to about 70% by weight of the total composition. Suitable flavoring agents may be present in the pharmaceutical composition to provide a cherry flavor, cotton candy flavor, or other suitable flavor to make the pharmaceutical composition easier for a human to ingest. The flavoring agent or mixtures thereof are typically present in an amount of about 0.0001% to about 5% by weight of the total composition.

Suitable preservatives include, for example, methylparaben, propylparaben, sodium benzoate, and benzalkoniyum chloride. A mixture of two or more preservatives may be used. The preservative or mixtures thereof are typically present in an amount of about 0.0001% to about 2% by weight of the total composition.

Suitable buffering agents include, for example, citric acid, sodium citrate, phosphoric acid, potassium phosphate, and various other acids and salts. A mixture of two or more buffering agents may be used. The buffering agent or mixtures thereof are typically present in an amount of about 0.001% to about 4% by weight of the total composition.

Suitable solvents for a liquid solution or suspension include, for example, sorbitol, glycerin, propylene glycol, and water. A mixture of two or more solvents may be used. The solvent or solvent system is typically present in an amount of about 1% to about 90% by weight of the total composition.

The pharmaceutical composition may be co-administered with adjuvants. For example, nonionic surfactants such as polyoxyethylene oleyl ether and n-hexadecyl polyethylene ether may be administered with or incorporated into the pharmaceutical composition to artificially increase the permeability of the intestinal walls. Enzymatic inhibitors may also be administered with or incorporated into the pharmaceutical composition.

### The Compound

In one embodiment of the invention a dose of 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 85±5 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 85±2 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 150±25 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 150±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 175±25 mg of 6-(3-chloro-2-fluorobenzyl)-1-[(2S)-1-hydroxy-3-methylbutan-2-yl]-7-methoxy-4-dihydroquinoline-3-carboxylic acid or a pharmaceutically acceptable salt thereof, is administered.

In one embodiment of the invention a dose of 175±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 170±25 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 170±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 300±50 mg of 6(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 300±20 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

In one embodiment of the invention a dose of 300±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is administered.

Compounds that Inhibit a UGT Pathway or UGT Metabolism

Uridine 5′-diphospho-glucuronosyltransferase (UDP-glucurono-syltransferase, UGT) based, Phase 2 metabolism via conjugation reactions (glucuronidation) represents one of a number of important primary or secondary metabolic pathways for endogenous and exogenously administered molecules, including many drugs. Glucuronidation reactions results in increased water solubility of the metabolized drug that facilitates its elimination by the body. While drug-drug interactions via the UGT pathway are less common and typically of a smaller magnitude than cytochrome P450-based interactions, inhibition of this pathway can result in clinically meaningful changes in the pharmacokinetics of molecules subject to this route of metabolism. Inhibition of this pathway can result in increased systemic exposures to drugs that could increase potency and/or adverse events. Induction of this pathway could result in lower exposures that may compromise efficacy of the drug of interest.

A number of compounds have been shown to inhibit UGT activity in humans. These include natural products (flavonoids), fatty acids, steroids, benzodiazepines, and non-steroidal anti-inflammatory drugs. See for Example Grancharov, K. et al., Pharmacology and Therapeutics, 2001, 89, 171-186; Kiang, T. K. L. et al., Pharmacology and Therapeutics, 2005, 106, 97-132; Williams, J. A. et al., Drug Metabolism and Disposition, 2004, 32, 1201-1208; and International Patent Application Publication Number WO/2003/055494.

One specific agent that blocks UGT activity and that is useful in the methods of the invention is atazanavir (1[4-(pyridine-2-yl)-phenyl]-4(S)-2,5-bis-[N-(N-methoxycarbonyl-(L)-tert-leucyl)amino]-6-phenyl-2-axahexane, or a pharmaceutically acceptable salt thereof), which is discussed in U.S. Pat. Nos. 5,849,911 and 6,087,783. Commercially available Reyataz (atazanavir sulfate) (2,56,10,13-pentaazatetradecanedioic acid, 3-12-bis(1,1-dimethylethyl)-8-hydroxy-4,11-dioxo-9-(phenylmethyl)-6-((-4-(2-pyridinyl)phenyl)methyl), dim ethyl ester, (3S,8S,9S,12S) sulfate (1:1) (salt) was used in the Examples below.

A specific dose of atazanavir that can be used according to the invention is 400±150 mg of atazanavir or a pharmaceutically acceptable salt thereof. A specific dose of atazanavir that can be used according to the invention is 400±100 mg of atazanavir or a pharmaceutically acceptable salt thereof. A specific dose of atazanavir that can be used according to the invention is 300±100 mg of atazanavir or a pharmaceutically acceptable salt thereof. A specific dose of atazanavir that can be used according to the invention, is 300±10 mg of atazanavir or a pharmaceutically acceptable salt thereof.

Certain compounds that inhibit a UGT pathway or UGT metabolism may also inhibit Cytochrome P-450. Accordingly, the term “compounds that inhibit a UGT pathway or UGT metabolism” includes such compounds.

Compounds that Inhibit Cytochrome P-450

As used herein, “Compounds that inhibit cytochrome P-450” include compounds that decrease the metabolism of Compound 1 by cytochrome P450, in particular, the metabolism of Compound 1 by cytochrome P450 3A. Accordingly, the term includes inhibitors of cytochrome P450, as well as substrates for cytochrome P450 and other compounds that decrease the metabolism of Compound 1 by cytochrome P450. A number of such compounds are known: see for example http://medicine.iupui.edu/flockhart/table.htm; and International Patent Application Publication Number WO 2008/010921.

Representative compounds include, cimetidine, fluoroquinolones, fluvoxamine, ticlopidine, thiotepa, ticlopidine, gemfibrozil, montelukast, fluoxetine, fluvoxamine, ketoconazole, lansoprazole, omeprazole, ticlopidine, amiodarone, fluconazole, isoniazid, amiodarone, buproprion, chlorpheniramine, cimetidine, clomipramine, duloxetine, fluoxetine, haloperidol, methadone, mibefradil, paroxetine, quinidine, ritonavir, disulfiram, indinavir, nelfinavir, amiodarone, cimetidine, clarithromycin, diltiazem, erythromycin, fluvoxamine itraconazole, ketoconazole, mibefradil, nefazodone, troleandomycin, and verapamil.

A specific sub-set of cytochrome P-450 inhibitors that are useful in the methods of the invention includes ketoconazole, itraconazole, clarithromycin, telithromycin, indinavir, nelfinavir, saquinavir, nefazadone, erythromycin and ritonavir, and pharmaceutically acceptable salts thereof.

Another specific sub-set of cytochrome P-450 inhibitors that are useful in the methods of the invention includes the HIV protease inhibitors indinavir, nelfinavir, saquinavir, and ritonavir.

One specific agent that blocks Cytochrome P-450 activity and that is useful in the methods of the invention is ritonavir, or a pharmaceutically acceptable salt thereof. A specific dose of ritonavir that can be used according to the invention is 100±50 mg of ritonavir or a pharmaceutically acceptable salt thereof. A specific dose of ritonavir that can be used according to the invention is 100±25 mg of ritonavir or a pharmaceutically acceptable salt thereof. A specific dose of ritonavir that can be used according to the invention is 100±10 mg of ritonavir or a pharmaceutically acceptable salt thereof.

Other specific agents that block Cytochrome P-450 activity and that are useful in the methods of the invention are reported in International Patent Application Publication Number WO 2008/010921. In one specific embodiment of the invention, the compound that inhibits cytochrome P-450 is a compound of the following formula:

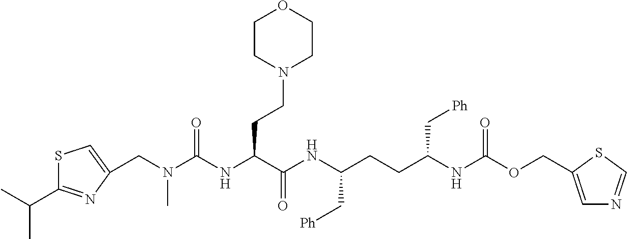 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof.

Specific embodiments described herein are for illustration and they do not exclude other defined values or other values within defined ranges.

### SPECIFIC EMBODIMENTS OF THE INVENTION

### Specific Embodiment 1

In one embodiment the invention provides a method of treating a viral infection in a human comprising administering 1) 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; and 2) a compound that inhibits a UGT pathway or UGT metabolism, or a pharmaceutically acceptable salt thereof to the human.

### Specific Embodiment 2

In one embodiment the invention provides the method of specific embodiment 1 wherein 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, are administered.

### Specific Embodiment 3

In one embodiment the invention provides the method of specific embodiment 1 wherein 150±25 mg of 6-(3-chloro-2-fluorobenzyl)-1-[(2S)-1-hydroxy-3-methylbutan-2-yl]-7-methoxy-4-oxo-14-dihydroquinoline-3-carboxylic acid or a pharmaceutically acceptable salt thereof, are administered.

### Specific Embodiment 4

In one embodiment the invention provides the method of specific embodiment 1 Wherein 300±50 mg of 6(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, are administered.

### Specific Embodiment 5

In one embodiment the invention provides the method of specific embodiment 1 wherein the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the compound that inhibits a UGT pathway or UGT metabolism, or a pharmaceutically acceptable salt thereof are coadministered.

### Specific Embodiment 6

In one embodiment the invention provides the method of any one of specific embodiments 1-4 wherein a single dosage form comprising the 6-(3-chloro-2-fluorobenzyl)-1-[(2S)-1-hydroxy-3-methylbutan-2-yl]-7-methoxy-4-oxo-1,4- dihydroquinoline-3-carboxylic acid or a pharmaceutically acceptable salt thereof, and the compound that inhibits a UGT pathway or UGT metabolism, or a pharmaceutically acceptable salt thereof is administered.

### Specific Embodiment 7

In one embodiment the invention provides the method of any one of specific embodiments 1-6 further comprising administering a compound that inhibits cytochrome P-450, or a pharmaceutically acceptable salt thereof to the human.

### Specific Embodiment 8

In one embodiment the invention provides the method of specific embodiment 7 wherein the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the compound that inhibits cytochrome P-450, or a pharmaceutically acceptable salt thereof are co-administered.

### Specific Embodiment 9

In one embodiment the invention provides the method of specific embodiment 7 wherein a single dosage form comprising the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the compound that inhibits cytochrome P-450, or a pharmaceutically acceptable salt thereof is administered.

### Specific Embodiment 10

In one embodiment the invention provides the method of specific embodiment 7 wherein the compound that inhibits a UGT pathway or UGT metabolism, or a pharmaceutically acceptable salt thereof, and the compound that inhibits cytochrome P-450, or a pharmaceutically acceptable salt thereof are co-administered.

### Specific Embodiment 11

In one embodiment the invention provides the method of specific embodiment 7 wherein a single dosage form comprising the compound that inhibits a UGT pathway or UGT metabolism, or a pharmaceutically acceptable salt thereof, and the compound that inhibits cytochrome P-450, or a pharmaceutically acceptable salt thereof is administered.

### Specific Embodiment 12

In one embodiment the invention provides the method of any one of specific embodiments 1-11 wherein the compound that inhibits a UGT pathway or UGT metabolism is a flavonoid, fatty acid, steroid, benzodiazepine, non-steroidal anti-inflammatory, or atazanavir, or a pharmaceutically acceptable salt thereof.

### Specific Embodiment 13

In one embodiment the invention provides the method of any one of specific embodiments 1-11 where in the compound that inhibits a UGT pathway or UGT metabolism is atazanavir or a pharmaceutically acceptable salt thereof.

### Specific Embodiment 14

In one embodiment the invention provides the method of specific embodiment 13 wherein 300±150 mg of atazanavir or a pharmaceutically acceptable salt thereof is administered.

### Specific Embodiment 15

In one embodiment the invention provides the method of any one of specific embodiments 7-14 wherein the compound that inhibits cytochrome P-450 is selected from ketoconazole, itraconazole, clarithromycin, telithromycin, indinavir, nelfinavir, saquinavir, nefazadone, erythromycin and ritonavir, and pharmaceutically acceptable salts thereof.

### Specific Embodiment 16

In one embodiment the invention provides the method of any one of specific embodiments 7-14 Wherein the compound that inhibits cytochrome P450 is a compound of the following formula:

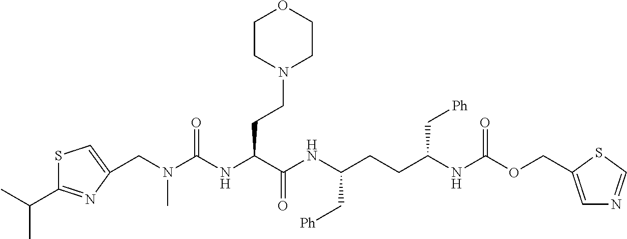 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof.

### Specific Embodiment 17

In one embodiment the invention provides the method of any one of specific embodiments 7-14 wherein the compound that inhibits cytochrome P450 is ritonavir, or a pharmaceutically acceptable salt thereof.

### Specific Embodiment 18

In one embodiment the invention provides the method of specific embodiment 17 wherein 100±50 mg of ritonavir or a pharmaceutically acceptable salt thereof is administered to the human.

### Specific Embodiment 19

In one embodiment the invention provides the method of any one of specific embodiments 1-18 wherein the virus is human immunodeficiency virus (HIV).

### Specific Embodiment 20

In one embodiment the invention provides a composition comprising 85±10 mg of 6(3-chloro-2-fluorobenzyl)-1[(2S)-1-hydroxy-3-methylbutan-2-yl]-7-methoxy-4-oxo-1,4-dihydroquinoline-3-carboxylic acid or a pharmaceutically acceptable salt thereof; a compound that inhibits a UGT pathway or UGT metabolism, or a pharmaceutically acceptable salt thereof; and a pharmaceutically acceptable carrier or diluent.

### Specific Embodiment 21

In one embodiment the invention provides a composition comprising 175±25 mg of 6(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; a compound that inhibits a UGT pathway or UGT metabolism, or a pharmaceutically acceptable salt thereof; and a pharmaceutically acceptable carrier or diluent.

### Specific Embodiment 22

In one embodiment the invention provides the composition of specific embodiment 20 or 21 wherein the compound that inhibits a UGT pathway or UGT metabolism is a flavonoid, fatty acid, steroid, benzodiazepine, non-steroidal anti-inflammatory, or atazanavir, or a pharmaceutically acceptable salt thereof.

### Specific Embodiment 23

In one embodiment the invention provides the composition of specific embodiment 20 or 21 where in the compound that inhibits a UGT pathway or UGT metabolism is atazanavir or a pharmaceutically acceptable salt thereof.

### Specific Embodiment 24

In one embodiment the invention provides the composition of specific embodiment 23 which comprise 300±150 to of atazanavir or a pharmaceutically acceptable salt thereof.

### Specific Embodiment 25

In one embodiment the invention provides the composition of any one of specific embodiments 20-24 which further comprises a compound that inhibits cytochrome P-450, or a pharmaceutically acceptable salt thereof.

### Specific Embodiment 26

In one embodiment the invention provides the composition of specific embodiment 25 wherein the compound that inhibits cytochrome P-450 is selected from ketoconazole, itraconazole, clarithromycin, telithromycin, indinavir, nelfinavir, saquinavir, nefazadone, erythromycin and ritonavir, and pharmaceutically acceptable salts thereof.

### Specific Embodiment 27

In one embodiment the invention provides the composition of specific embodiment 25 wherein the compound that inhibits cytochrome P-450 is ritonavir, or a pharmaceutically acceptable salt thereof.

### Specific Embodiment 28

In one embodiment the invention provides the composition of specific embodiment 27 which comprises 100±50 mg of ritonavir or a pharmaceutically acceptable salt thereof.

### Specific Embodiment 29

In one embodiment the invention provides the composition of specific embodiment 25 wherein the compound that inhibits cytochrome P-450 is a compound of the following formula:

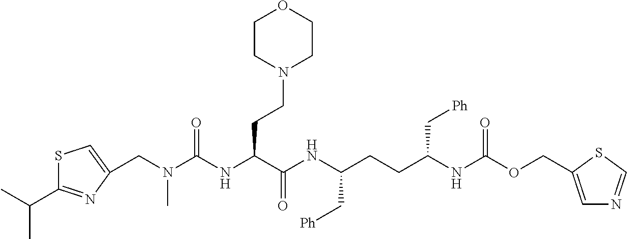 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof.

### Specific Embodiment 30

In one embodiment the invention provides a kit comprising: (1) a unit dosage form comprising 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof; (2) a compound that Inhibits a UGT pathway or UGT metabolism, or a pharmaceutically acceptable salt thereof; (3) one or more containers; and (4) prescribing information regarding administering the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof with the compound that inhibits a UGT pathway or UGT metabolism, or the pharmaceutically acceptable salt thereof.

### Specific Embodiment 31

In one embodiment the invention provides the kit of specific embodiment 30 wherein the unit dosage form comprises 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

### Specific Embodiment 32

In one embodiment the invention provides the kit of specific embodiment 30 wherein the unit dosage form comprises 175±25 mg of 6-(3-chloro-2-flourobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

### Specific Embodiment 33

In one embodiment the invention provides the kit of specific embodiment 30 wherein the compound that inhibits a UGT pathway or UGT metabolism is a flavonoid, fatty acid, steroid, benzodiazepine, non-steroidal anti-inflammatory, or atazanavir, or a pharmaceutically acceptable salt thereof.

### Specific Embodiment 34

In one embodiment the invention provides the kit of specific embodiment 30 wherein the compound that inhibits a UGT pathway or LICIT metabolism is atazanavir or a pharmaceutically acceptable salt thereof.

### Specific Embodiment 35

In one embodiment the invention provides the kit of specific embodiment 34 which comprises 400±150 mg of atazanavir or a pharmaceutically acceptable salt thereof.

### Specific Embodiment 36

In one embodiment the invention provides the kit of any one of specific embodiments 30-35 which further comprises a compound that inhibits cytochrome P-450, or a pharmaceutically acceptable salt thereof.

### Specific Embodiment 37

In one embodiment the invention provides the kit of specific embodiment 36 wherein the compound that inhibits cytochrome P-450 is selected from ketoconazole, itraconazole, clarithromycin, telithromycin, indinavir, nelfinavir saquinavir, nefazadone, erythromycin and ritonavir, and pharmaceutically acceptable salts thereof.

### Specific Embodiment 38

In one embodiment the invention provides the kit of specific embodiment 36 wherein the compound that inhibits cytochrome P-450 is ritonavir, or a pharmaceutically acceptable salt thereof.

### Specific Embodiment 39

In one embodiment the invention provides the kit of specific embodiment 38 which comprises 100±50 mg of ritonavir or a pharmaceutically acceptable salt thereof.

### Specific Embodiment 40

In one embodiment the invention provides the kit of specific embodiment. 36 wherein the compound that inhibits cytochrome P-450 is a compound of the following formula:

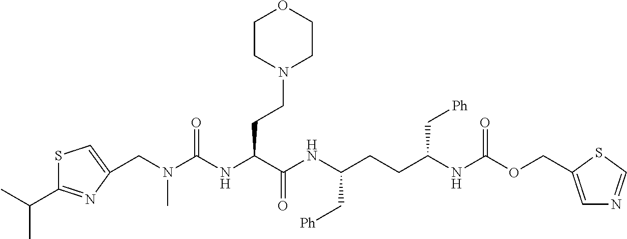 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof.

In one specific embodiment the invention comprises administering about 85 mg (e.g. ±10 mg, 5 mg, or 2 mg) of the Compound, or a pharmaceutically acceptable salt thereof in another specific embodiment, the invention comprises administering about 175 mg (e.g. ±25 mg or 10 mg) of the Compound, or a pharmaceutically acceptable salt thereof. In another specific embodiment, the invention comprises administering about 170 nig (e.g. ±25 mg or 10 mg) of the Compound, or a pharmaceutically acceptable salt thereof.

In one specific embodiment, the invention comprises administering about 300 mg (e.g. ±150 mg, 100 mg, 50 mg, or 10 mg) of atazanavir, or a pharmaceutically acceptable salt thereof.

In one specific embodiment, the invention provides a kit comprising: (1) a unit dosage form comprising 85 mg±10 mg, 6-(3-chloro-2-fluorobenzyl)-1-[(2S)-1-hydroxy-3-methylbutan-2-yl]-7-methoxy-4-oxo-4,4-dihydroquinoline-3-carboxylic acid, or a pharmaceutically acceptable salt thereof; (2) a compound that inhibits a UGT pathway or UGT metabolism (e.g. atazanavir), or a pharmaceutically acceptable salt thereof; (3) one or more containers; and (4) prescribing information regarding administering the 6-(3-chloro-2-fluorobenzyl)-1-[(2S)-hydroxy-3-methylbutan-2-yl]-7-methoxy-4-oxo-1,4-dihydroquinoline-3-carboxylic acid or a pharmaceutically acceptable salt thereof with the compound that inhibits a UGT pathway or UGT metabolism, or the pharmaceutically acceptable salt thereof.

In one specific embodiment, the invention provides a kit comprising: (1) a unit dosage form comprising 170 mg±25 mg 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof; (2) a compound that inhibits a UGT pathway or UGT metabolism (e.g. atazanavir), or a pharmaceutically acceptable salt thereof; (3) one or more containers; and (4) prescribing information regarding administering the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof with the compound that inhibits a UGT pathway or UGT metabolism or the pharmaceutically acceptable salt thereof.

In one specific embodiment, the invention provides a kit comprising: (1) a unit dosage form comprising 175 mg±25 mg 6-(3-chloro-2-flourobenzyl)1-[(2S)-1-hydroxy-3-methylbutan-2-yl]-7-methoxy-4-oxo-1,4-dihydroquinoline-3-carboxylic acid, or a pharmaceutically acceptable salt thereof; (2) a compound that inhibits a UGT pathway or UGT metabolism (e.g. atazanavir), or a pharmaceutically acceptable salt thereof; (3) one or more containers; and (4) prescribing information regarding administering the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof with the compound that inhibits a UGT pathway or UGT metabolism, or the pharmaceutically acceptable salt thereof.

The invention will now be illustrated by the following non-limiting Examples.

### EXAMPLES

### Example 1

### A Pharmacokinetic Interaction Between Atazanavir/r and the Compound

The effects of the coadministration of atazanavir/r (ATV/r) with the Compound were determined. This study evaluated the safety and steady-state pharmacokinetics of the coadministered Compound and ATV/r.

### Methods

Healthy subjects were randomized to follow one of six sequences to receive the Compound QD alone, ATV/r alone (300/100 mg QD) and the Compound+ATV/r QD each for fourteen days, The Compound doses were 200 mg in study 1 and 150 mg (reference) and 85 mg in study 2. Lack of PK alteration bounds for 90% confidence intervals (CI) about the geometric mean ratio (GMR) (coadministration: alone) were 70-143% for the Compound and 70-125% for ATV as no dose adjustments were recommended upon 30% lower exposures by other drugs.

### Results

In study 1, 3361 and study 2, 19/30 subjects completed each study; discontinuations were mostly for known ATV adverse events. Pharmacokinetic results were as follows:

% GMR (90% CI)

| Study 1 | the Compound (n = 33)^{a} | ATV (n = 33) |
|---|---|---|
| AUCtau | 200 (185, 216) | 79.2 (73.6, 853) |
| Cₘₐₓ | 185 (169, 203) | 84.3 (78.2, 90.9) |
| Cₜₐᵤ | 288 (253, 327) | 65.5 (59.1, 72.6) |
| Study 2 | the Compound (n = 20)^{b} | ATV (n = 20) |
| AUCₜₐᵤ | 107 (95.1, 121) | 88.6 (79.6, 98.7) |
| Cₘₐₓ | 90.9 (81.4, 102) | 96.0 (86.1, 107) |
| Cₜₐᵤ | 138 (118, 163) | 82.6 (71.9, 94.9) |

^{a}test and reference 200 mg
^{b}test 85 mg, reference 150 mg

The Compound exposures were elevated upon coadministration with ATV/r, likely via inhibition of UGT1A1/3 metabolism in addition to inhibition of CYP3A.

A reduced dose of the Compound was selected through modeling a variety of doses using compartmental modeling in WinNonlin (Pharsight Corporation, Mountain View, Calif., USA) incorporating the observed drug-drug interaction data with atazanavir from study 1. Consideration was given to achieving equivalent Compound exposures in patients receiving and not receiving atazanavir using pharmacokinetic (bio-) equivalence comparisons (Pharsight Corporation, Mountain View, Calif., USA). Consideration was also given to minimizing the number of individuals with extreme outliers in (low or high) exposures. This modeling was subsequently validated in a clinical study that established that this dose reduction resulted in equivalent Cₘₐₓ and AUC for the Compound when the reduced dose was coadministered with atazanavir/r. Due to the fact that this interaction manifests as a more pronounced effect on trough (Cₜₐᵤ) concentrations, this lower dose continues to provide high trough concentrations while limiting unnecessary high systemic exposures to the Compound. Thus, the 85 mg and 150 mg doses of the Compound with atizanavir/r are expected to provide similar systemic exposures (AUC) to the 150 mg and 300 mg ritonavir-boosted doses without atazanavir, ATV exposures were modestly lower with 200 mg of the Compound and not affected with the reduced 85 mg dose. Accordingly, a reduction of about 40-60% in the dose of the Compound can be administered with atazanavir while maintaining an equivalent exposure.

### Conclusion

A reduced dose of the Compound (e.g. 85±10 mg) can be administered with atazanavir and ritonavir to achieve a comparable systemic exposure to a higher dose when the Compound is administered with only ritonavir. It is believed that atazanavir improves the pharmacokinetic exposure of the Compound by blocking the UGT1A1/3 metabolic pathway of the Compound.

Similar studies were carried out determining the effect of five different protease inhibitors on the pharmacokinetics of the Compound. These studies employed various doses of ritonavir (100 mg QD to 200 mg BID). Of the five protease inhibitors that were tested, three were found to have no effect on the pharmacokinetics of the Compound. Only two (including atazanavir) of the five were found to have an improved pharmacokinetic effect on the Compound.

### Example 2

### A Pharmacokinetic Interaction Between Atazanavir and the Compound

The effects of the coadministration of atazanavir (ATV) with the Compound were determined. This study evaluated the safety and steady-state pharmacokinetics of the co-administered Compound and ATV.

### Background

Elvitegravir (EVG), an HIV integrase inhibitor metabolized primarily via CYP3A and glucuronidation, displays substantially higher systemic levels (boosting) when coadministered with low doses of the potent. mechanism-based CYP3A inhibitor ritonavir (EVG/r). This study explored the ability of atazanavir (ATV), another strong yet less potent CYP3A inhibitor that also inhibits UGT-mediated metabolism, to boost the plasma exposure of EVCI.

### Methods

Healthy subjects received EVG/r 300/100 mg or EVG/ATV 300/400 mg in a randomized, crossover manner for 10 days each, with the last dose being coadministered with the CYP3A probe substrate midazolam (oral syrup; 5 mg) and pharmacokinetic (PK) sampling performed, Elvitegravir exposures (dosed with ATV versus RTV) were evaluated, using 90% confidence interval (CI) hounds of 1) 80 to 125% to establish equivalent boosting or 2) 60-167% to establish non-inferior EVG exposures versus those observed in Phase 2 and planned Phase 3 studies for the PK parameters AUCₜₐᵤ, Cₘₐₓ, and Cₜₐᵤ. ATV, midazolam, and ritonavir pharmacokinetics were also determined for descriptive statistics.

### Results

Fifteen of the 18 enrolled subjects completed the study 2 subjects discontinued due to adverse events (AE), one each due to rash (receiving EVG/ATV) and elevated creatinine phosphokinase (receiving EVG/r) No grade 4 AE or serious AE were observed. For the PK analysis set (n=14 due to one outlier), the geometric mean ratio (90% CI) (EVG/ATV vs. EVG/r) for EVG Cₘₐₓ, AUCₜₐᵤ, Cₜₐᵤ were 108 (99, 119), 107 (95.6, 119), 89.9 (71.4, 113) and median EVG T_{1/2} was 5.2 versus 6.3 hours. Corresponding values for midazolam Cₘₐₓ, AUC₀₋ₗₐₛₜ, and AUC_{inf} were 98.8 (89,6, 109), 91.8, (83.3. 101), and 89.5 (80.7, 99.3), suggesting similar CYP3A effects of ATV 400 and WIN 100 mg when coadministered with EVG. Consistent with the inhibition of UGT1A1 by ATV, mean AUCₜₐᵤ of GS-9200, the UGT1A1/3-mediated glucuronide metabolite of EVG, were 37% lower with EVG/ATV dosing versus EVG/r. Mean (% CV) atazanavir AUCₜₐᵤ and Cₜₐᵤ were 16300(29.5%) ng·hr/ml and 74.5 (45.7%) ng/ml, respectively, and lower than historical data.

### Conclusions

Once-daily atazanavir has the potential to boost EVG via inhibition, of CYP3 A-mediated metabolism similar to that by ritonavir.

### Example 3

### Representative Example of the Formulations of 6-(3-chloro-2-fluorobenzyl)1-[(2S)-1-hydroxy-3-methylbutan-2-yl]-7-methoxy-4-oxo1,4-dihydroquinoline-3-carboxylic acid

TABLE 1
200 mg Tablets

| Component | Function | Amount Per Tablet |
|---|---|---|
| The Compound | Drug substance | 200.0 mg |
| Mannitol USP | Diluent | 107.6 mg |
| Colloidal Silicon Dioxide NF | Glidant | 25.0 mg |
| Sodium lauryl sulfate NF | Surfactant | 10.0 mg |
| Crospovidone NF | Disintegrant | 25.0 mg |
| Hypromellose 2910 USP | Binder | 26.0 mg |
| Purified water*¹ USP | Binder agent | — |
| Croscarmellose sodium NT | Disintegrant | 100.0 mg |
| Magnesium Stearate NF | Lubricant | 2.4 mg |
| Total tablet weight | | 490.0 mg |

*¹The purified water is removed during processing.

The Compound was first micronized with a jet mill. The micronized compound was mixed with Mannitol, Crospovidone, and Colloidal Silicon Dioxide in a polyethylene (PE) bag and then passed though a 500 μm screen three times. Hypromellose 2910 was separately dissolved in purified water by stirring and sodium lauryl sulfate was added and dissolved. The Mannitol/Crospovidone/Colloidal Silicon Dioxide/the Compound mixture was placed in a fluidized-bed granulator and was granulated using the Hypromellose/sodium lauryl sulfate solution. After granulation, the wet granulates were dried in the same granulator, I be dried granules were passed through a 500 μm screen.

The screened granules were then mixed with croscarmellos sodium in a blender and magnesium stearate was added to the blender and mixed. The granules were compressed into tablets using a rotary tableting machine.

TABLE 2
85 mg Tablets

| Component | Function | Amount Per Tablet |
|---|---|---|
| The Compound | Drug substance | 85.0 mg |
| Lactose Monohydrate NF | Diluent | 6.2 mg |
| Microcrystalline Cellulose NF | Diluent | 67.1 mg |
| Sodium lauryl sulfate NF | Surfactant | 6.4 mg |
| Croscarmellose sodium NF | Disintegrant | 19.1 mg |
| Hydroxypropyl Cellulose NF | Binder | 4.2 mg |
| Purified water*¹ USP | Binder agent | — |
| Microcrystaline Cellulose NF | Diluent | 17.0 mg |
| Croscarmellose sodium NF | Disintegrant | 6.4 mg |
| Magnesium Stearate NF | Lubricant | 1.1 mg |
| Total tablet weight | | 212.5 mg |

*¹The purified water is removed during processing.

The Compound was first micronized with a jet mill. The micronized compound was mixed with Lactose Monohydrate Microcrystalline Cellulose, and Croscarmellose sodium in a fluid bed granulator. Hydroxypropyl Cellulose was separately dissolved in purified water by stirring and sodium lauryl sulfate was added and dissolved. The Lactose Monohydrate/Microcrystalline Cellulose/Croscarmellose sodium/the Compound mixture was granulated in the fluid-bed granulator using the Hydroxypropyl cellulose/sodium lauryl sulfate solution. After granulation, the wet granulates were dried in the same granulator. The dried granules were passed through a 500 μm screen.

The screened granules were then mixed with Microcrystalline Cellulose and Croscarmellos sodium in a blender and Magnesium stearate was added to the blender and mixed. The granules were compressed into tablets using a rotary tableting machine.

### Example 4

### Representative Examples of the Formulations of 6-(3-chloro-2-fluorobenzyl)-1-[(2S)-1-hydroxy-3-methylbutan-2-yl]-7-methoxy-4-oxo-1,4-dihydroquinoline-3-carboxylic acid (The Compound)

| | Amount | | | |
|---|---|---|---|---|
| Components | (% w/w) | 85 mg | 150 mg | 300 mg |
| The Compound (micronized) | 40.0 | 85.0 | 150.0 | 300.0 |
| Lactose Monohydrate, NF | 2.9 | 6.2 | 10.9 | 215.7 |
| (Fast Flo 316) | | | | |
| Microcrystalline Cellulose, NF | 39.35 | 83.6 | 147.6 | 295.1 |
| (Avicel PH-101) | | | | |
| Hydroxypropyl Cellulose, NF | 2.0 | 4.2 | 7.5 | 15.0 |
| (Klucel EF) | | | | |
| Sodium Lauryl Sulfate, NF | 3.0 | 6.4 | 11.2 | 22.5 |
| (Stepanol WA-100) | | | | |
| Croscarmellose Sodium, NF | 12.0 | 25.5 | 45.0 | 90.0 |
| (Ac-Di-Sol) | | | | |
| Magnesium Stearate, NF | 0.75 | 1.6 | 2.8 | 5.7 |
| (Code 5712) | | | | |
| Purified Water, USP | — | — | — | — |
| Total | 100.0 | 212.5 | 3750 | 750.0 |
| Film-Coating Composition: | | | | |
| Opadry II Green 85F91203 | — | 6.4 | 11.3 | 22.5 |
| Purified Water, USP | Not | — | — | — |
| | Specified | | | |

All references including publications, patent applications, and patents, cited herein are hereby incorporated by reference in their entirety.

The use of the terms “a” and “an” and “the” and similar referents in the context of describing the invention (including the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range. unless otherwise indicated herein, and each separate value is incorporated into the specification as if it were individually recited herein. All. methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The embodiments within the specification provide an illustration of embodiments of the invention, and should not be construed to limit the scope of the invention. The skilled artisan recognizes that many other embodiments are encompassed by the claimed invention and that it is intended that the specification and examples be considered as exemplary only, with the true scope and spirit of the invention being indicated by the following claims.

1. (canceled)

2. The composition of claim 9 characterized in that the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the atazanavir, or a pharmaceutically acceptable salt thereof are coadministered.

3. The composition of claim 9 characterized in that the composition is in a single dosage form comprising the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the atazanavir, or a pharmaceutically acceptable salt thereof.

4. The composition of claim 9 characterized in that the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the ritonavir, or a pharmaceutically acceptable salt thereof are co-administered.

5. The composition of claim 9 characterized in that the composition is in a single dosage form comprising the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the ritonavir, or a pharmaceutically acceptable salt thereof.

6. The composition of claim 9 characterized in that the atazanavir, or a pharmaceutically acceptable salt thereof, and the ritonavir, or a pharmaceutically acceptable salt thereof are co-administered.

7. The composition of claim 9 characterized in that the composition is in a single dosage form comprising the atazanavir, or a pharmaceutically acceptable salt thereof, and the ritonavir, or a pharmaceutically acceptable salt thereof.

8. (canceled)

9. A composition for treating an infection of human immunodeficiency virus (HIV) in a human comprising 85±10 mg per day of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; 300±150 mg per day of atazanavir, or a pharmaceutically acceptable salt thereof; 100±50 mg per day of ritonavir or a pharmaceutically acceptable salt thereof; and a pharmaceutically acceptable carrier or diluent.

10. A composition for treating an infection of human immunodeficiency virus (HIV) in a human comprising 85 ±10 mg per day of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; 300±150 mg per day of atazanavir, or a pharmaceutically acceptable salt thereof; a compound of the following formula: 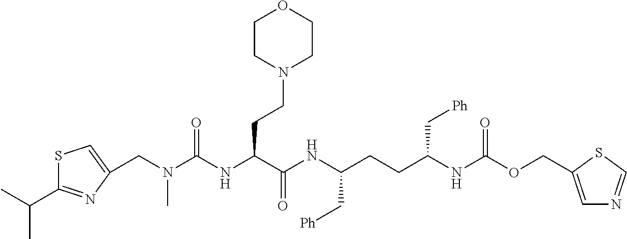 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]
or a pharmaceutically acceptable salt thereof; and a pharmaceutically acceptable carrier or diluent.

11-12. (canceled)

13. A method of treating an infection of human immunodeficiency virus (HIV) in a human, comprising administering the composition of claim 9 to the human, characterized in that the 6-(3-chloro-2-fluorobenzyl)-1-[(2S)-1- hydroxy-3-methylbutan-2-yl]-7-methoxy-4-oxo-1,4-dihydroquinoline-3-carboxylic acid or the pharmaceutically acceptable salt thereof, is administered in conjunction with 300 mg per day of atazanavir, or a pharmaceutically acceptable salt thereof to the human.

14. The method of claim 13 characterized in that the 6-(3-chloro-2-fluorobenzyl)-1-[(2S)-1- hydroxy-3-methylbutan-2-yl]-7-methoxy-4-oxo-1,4-dihydroquinoline-3-carboxylic acid or the pharmaceutically acceptable salt thereof, is administered in conjunction with 300 mg per day of atazanavir, or a pharmaceutically acceptable salt thereof; and with 100 mg per day of ritonavir, or a pharmaceutically acceptable salt thereof, to the human.

15. The method of claim 18, wherein the method comprises administering 300 mg per day of atazanavir, or a pharmaceutically acceptable salt thereof, in combination with 85 mg per day of 6-(3-chloro-2-fluorobenzyl)-1-[(2S)-1- hydroxy-3-methylbutan-2-yl]-7-methoxy-4-oxo-1,4-dihydroquinoline-3-carboxylic acid, or a pharmaceutically acceptable salt thereof, to a human.

16. A method of inhibiting activity of a retroviral integrase in a human comprising administering the composition of claim 9 to the human characterized in that the 6-(3-chloro-2-fluorobenzyl)-1-[(2S)-1- hydroxy-3-methylbutan-2-yl]-7-methoxy-4-oxo-1,4-dihydroquinoline-3-carboxylic acid or a pharmaceutically acceptable salt thereof, is administered in conjunction with 300 mg per day of atazanavir, or a pharmaceutically acceptable salt thereof to the human.

17. The method of claim 16, further characterized in that the 6-(3-chloro-2-fluorobenzyl)-1-[(2S)-1- hydroxy-3-methylbutan-2-yl]-7-methoxy-4-oxo-1,4-dihydroquinoline-3-carboxylic acid or a pharmaceutically acceptable salt thereof, is administered in conjunction with 300 mg per day of atazanavir, or a pharmaceutically acceptable salt thereof, and with 100 mg per day of ritonavir, or a pharmaceutically acceptable salt thereof to the human.

18. A method of treating an infection of human immunodeficiency virus (HIV) in a human, comprising administering 300±150 mg per day of atazanavir, or a pharmaceutically acceptable salt thereof; in combination with 85±10 mg per day of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof, to the human.

19. The method of claim 18, further comprising administering 100±50 mg per day of ritonavir, or a pharmaceutically acceptable salt thereof, in combination with the 85±10 mg per day of 6-(3-chloro-2-fluorobenzyl)-1- [(2S)-1-hydroxy-3-methylbutan-2-yl]-7-methoxy-4- oxo-1,4-dihydroquinoline-3-carboxylic acid, or a pharmaceutically acceptable salt thereof, to the human.

20-21. (canceled)

22. The composition of claim 9, wherein the 85 mg per day of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is used in combination with 300 mg per day of atazanavir, or a pharmaceutically acceptable salt thereof.

23. The composition of claim 9, further comprising (c) 100 mg per day of ritonavir, or a pharmaceutically acceptable salt thereof.

24. (canceled)

25. The composition of claim 23, wherein the 85 mg per day of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, is used in combination with 300 mg per day of atazanavir, or a pharmaceutically acceptable salt thereof; and 100 mg per day of ritonavir, or a pharmaceutically acceptable salt thereof.

26. (canceled)

27. The composition of claim 9, wherein the composition comprises 85 mg per day of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; 300 mg per day of atazanavir, or a pharmaceutically acceptable salt thereof; 100 mg per day of ritonavir or a pharmaceutically acceptable salt thereof; and a pharmaceutically acceptable carrier or diluent.

28. (canceled)
